(19) United States
(12) Patent Application Publication
(10) Pub. No.: US 2009/0247546 A1
(43) Pub. Date: Oct. 1, 2009

(54) Treatment of Prevention of Valvular Heart Disease with Flibanserin

(75) Inventors: Angelo Ceci, Mittelbiberach (DE); Peter Bette, Maselheim (DE)

Correspondence Address: Michael P. Morris;Boehringer Ingelheim USA Corporation, 900 Ridgebury Road, Ridgefield, CT, 06877-0368, US

(73) Assignee: BOEHRINGER INGELHEIM INTERNATIONAL GMBH, Ingelheim (DE)

(21) Appl. No.: 12/280,804

(22) PCT Filed: Feb. 26, 2007
(86) PCT No.: PCT/EP2007/051780

§ 371 Date: Nov. 12, 2008

(30) Foreign Application Priority Data
Feb. 28, 2006 (EP) 06004065.6

(51) Int. Cl. A61K 31/496; A61P 9/10

(52) U.S. Cl. 514/254.06

(57) ABSTRACT

The invention relates to a method for the treatment or prevention of Valvular Heart Disease comprising the administration of a therapeutically effective amount of flibanserin.

The invention relates to a method for the treatment or prevention of Valvular Heart Disease comprising the administration of a therapeutically effective amount of flibanserin.

### DESCRIPTION OF THE INVENTION

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

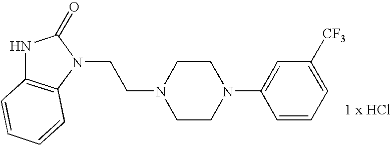

Flibanserin shows affinity for the 5-HT1A and 5-HT2-receptor. It is therefore a promising therapeutic agent for the treatment or prevention of a variety of diseases, for instance depression, schizophrenia, and anxiety.

Now, experiments provided evidence, that flibanserin can not only be used for the aforementioned diseases but also for the treatment or prevention of Valvular Heart Disease.

Within the present invention, the term “Valvular heart disease” relates to any dysfunction or abnormality of one or more of the heart's four valves, including the mitral valve and aortic valve of the left heart, and the tricuspid valve and pulmonic valve of the right heart. In a normally functioning heart, the four valves (flaps made of tissue) prevent blood from flowing backwards into the ventricles and while allowing the forward flow of blood into the lung and peripheral circulation in the course of the cardiac action.

According to the American Heart Association's 2004 Heart and Stroke Statistical Update, valvular heart disease is responsible for nearly 20,000 deaths each year in the United States and is a contributing factor in about 42,000 deaths. The majority of these cases involve disorders of the aortic valve (63 percent) and the mitral valve (14 percent). Deaths due to pulmonic and tricuspid valve disorders are more rare (0.06 percent and 0.01 percent, respectively). There are a number of types of valvular heart disease, including:

### a) Valvular Stenosis:

A condition in which there is a narrowing, stiffening, thickening, fusion or blockage of one or more valves of the heart. As a result, the defective valve can interfere with the smooth passage of blood through it leading to increased resistance. Depending on which valve is affected, the diagnosis may be aortic stenosis, mitral stenosis, pulmonic stenosis or tricuspid stenosis.

### b) Valvular Regurgitation:

A condition in which blood leaks backwards because one or more of the heart's valves is closing improperly. The nature and severity of the leakage, in turn, may increase the blood volume that is moved during each cardiac cycle or even keep the heart from circulating an adequate amount of blood. Depending on which valve is affected, the diagnosis may be aortic regurgitation, mitral regurgitation, pulmonary regurgitation or tricuspid regurgitation.

### c) Atresia of One of the Valves:

A serious condition in which one of the valves have failed to develop properly and is completely closed at birth. Depending on which valve is affected, the diagnosis may be aortic atresia, mitral atresia, pulmonary atresia or tricuspid atresia.

### d) Mitral Valve Prolapse:

A common and rarely serious condition in which the two flaps of the mitral valve (located between the left atrium and the left ventricle) can not close properly, and may result in blood leaking back into the left atrium (mitral valve regurgitation). It is due to either one (or both) of the flaps being too large, or because the muscle “hinges” of the flaps are too long

Accordingly, the instant invention relates to a method for the treatment or prevention of Valvular Heart Disease comprising the administration of a therapeutically effective amount of flibanserin, optionally in form the free base, the pharmacologically acceptable acid addition salts and/or optionally in form of the hydrates and/or solvates thereof.

In another embodiment, the instant invention relates to a method for the treatment or prevention of Valvular stenosis comprising the administration of a therapeutically effective amount of flibanserin, optionally in form the free base, the pharmacologically acceptable acid addition salts and/or optionally in form of the hydrates and/or solvates thereof.

In another embodiment, the instant invention relates to a method for the treatment or prevention of Valvular Regurgitation comprising the administration of a therapeutically effective amount of flibanserin, optionally in form the free base, the pharmacologically acceptable acid addition salts and/or optionally in form of the hydrates and/or solvates thereof.

In another embodiment, the instant invention relates to a method for the treatment or prevention of Atresia of one of the Valves comprising the administration of a therapeutically effective amount of flibanserin, optionally in form the free base, the pharmacologically acceptable acid addition salts and/or optionally in form of the hydrates and/or solvates thereof.

In another embodiment, the instant invention relates to a method for the treatment or prevention of Mitral Valve Prolapse comprising the administration of a therapeutically effective amount of flibanserin, optionally in form the free base, the pharmacologically acceptable acid addition salts and/or optionally in form of the hydrates and/or solvates thereof.

Another embodiment of the invention relates to the use of flibanserin, optionally in form the free base, the pharmacologically acceptable acid addition salts and/or optionally in form of the hydrates and/or solvates thereof, for the preparation of a medicament for the treatment or prevention of any of the aforementioned conditions.

Flibanserin can optionally be used in form of its pharmaceutically acceptable acid addition salts. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin is used in form of the free base, it is preferably used in form of flibanserin polymorph A as disclosed in WO 03/014079.

Flibanserin, optionally used in form the free base, the pharmacologically acceptable acid addition salts and/or optionally in form of the hydrates and/or solvates thereof, may be incorporated into the conventional pharmaceutical preparation in solid, liquid or spray form. The composition may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range applicable per day is between 0.1 to 400, preferably between 1.0 to 300, more preferably between 2 to 200 mg Flibanserin. Each dosage unit may conveniently contain from 0.01 mg to 100 mg Flibanserin, preferably from 0.1 to 50 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

### Examples of Pharmaceutical Formulations

| | | |
|---|---|---|
| A) | Tablets | per tablet |

| | |
|---|---|
| flibanserin hydrochloride | 100 mg |
| lactose | 240 mg |
| corn starch | 340 mg |
| polyvinylpyrrolidone | 45 mg |
| magnesium stearate | 15 mg |
| | 740 mg |

The finely ground active substance, lactose and some of the corn starch are mixed together. The mixture is screened, then moistened with a solution of Polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | flibanserin hydrochloride | 80 mg |
| | corn starch | 190 mg |
| | lactose | 55 mg |
| | microcrystalline cellulose | 35 mg |
| | polyvinylpyrrolidone | 15 mg |
| | sodium-carboxymethyl starch | 23 mg |
| | magnesium stearate | 2 mg |
| | | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| | | |
|---|---|---|
| C) | Coated tablets | per coated tablet |

| | | |
|---|---|---|
| flibanserin hydrochloride | 5 | mg |
| corn starch | 41.5 | mg |
| lactose | 30 | mg |
| polyvinylpyrrolidone | 3 | mg |
| magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| | | |
|---|---|---|
| D) | Capsules | per capsule |

| | | |
|---|---|---|
| Flibanserin hydrochloride | 150 | mg |
| Corn starch | 268.5 | mg |
| Magnesium stearate | 1.5 | mg |
| | 420 | mg |

The substance and corn starch are mixed and moistened with water. The moist mass is screened and dried. The dry granules are screened and mixed with magnesium stearate. The finished mixture is packed into size 1 hard gelatine capsules.

| | |
|---|---|
| E) | Ampoule solution |

| | | |
|---|---|---|
| flibanserin hydrochloride | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

| | |
|---|---|
| F) | Suppositories |

| | |
|---|---|
| flibanserin hydrochloride | 50 mg |
| solid fat | 1650 mg |
| | 1700 mg |

The hard fat is melted. At 40° C. the ground active substance is homogeneously dispersed. It is cooled to 38° C. and poured into slightly chilled suppository moulds.

In a particular preferred embodiment of the instant invention, flibanserin is administered in form of specific film coated tablets. Examples of these preferred formulations are listed below. The film coated tablets listed below can be manufactured according to procedures known in the art (see hereto WO 03/097058).

### G) Film Coated Tablet

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (e.g. Pharmacoat 606) | 1.250 |
| Carboxymethylcellulose sodium | 2.500 |
| Magnesium stearate | 0.625 |

Coating

| | |
|---|---|
| HPMC (e.g. Pharmacoat 606) | 1.400 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### H) Film Coated Tablet

| | |
|---|---|
| Constituents | mg/tablet |

Core

| | |
|---|---|
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### I) Film Coated Tablet

| | |
|---|---|
| Constituents | mg/tablet |

Core

| | |
|---|---|
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### J) Film Coated Tablet

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### K) Film Coated Tablet

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### L) Film Coated Tablet

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

1-8. (canceled)

9) A method for the treatment of Valvular Heart Disease comprising the administration of a therapeutically effective amount of flibanserin optionally in form the free base or a pharmacologically acceptable acid addition salts thereof.

10) A method according to claim 9 wherein the Valvular Heart Disease is a Valvular Stenosis.

11) A method according to claim 9 wherein the Valvular Heart Disease is a Valvular Regurgitation.

12) A method according to claim 9 wherein the Valvular Heart Disease is a Atresia of one of the Valves.

13) A method according to claim 9 wherein the Valvular Heart Disease is a Mitral Valve Prolapse.

14) A method according to claim 9, wherein flibanserin is administered in the form of a pharmaceutically acceptable acid addition salt wherein the salt is formed by an acid selected from the group consisting of succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

15) A method according to claim 9, wherein flibanserin is administered as a free base.

16) A method according to claim 9, wherein flibanserin is administered as a free base in form of its polymorph A.

17) A method according to claim 9, wherein flibanserin is administered in a dosage range between 0.1 to 400 mg per day.
